(19) United States
(12) Patent Application Publication
(10) Pub. No.: US 2005/0004068 A1
(43) Pub. Date: Jan. 6, 2005

(54) Modulation of tor

(76) Inventors: Patrick Brian Dennis, Fairborn, OH (US); Anja Jaeschke, Worcester, MA (US); Sara Kozma, Wyoming, OH (US); Masao Saitoh, Tokyo (JP); George Thomas, Wyoming, OH (US)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/491,945

(22) PCT Filed: Oct. 11, 2002
(86) PCT No.: PCT/EP02/11429

(30) Foreign Application Priority Data
Oct. 12, 2001 (GB) 0124577.8

(51) Int. Cl. C12Q 1/66; A61K 31/7076; A61K 31/522

(52) U.S. Cl. 514/46; 435/8; 514/263.3

(57) ABSTRACT

Methods for screening for potential modulators of mammalian target of rapamycin (mTOR) are provided based on detecting cellular ATP levels. Also provided are compositions useful in treating diseases or conditions dependent on mTOR signaling, including cancer, rheumatoid arthritis, restinosis and transplant rejection.

The present invention relates to the field of tumour and cancer therapy. More particularly, the invention relates to methods of screening agents for anti-tumour and/or anti-tumourigenic activity, and therapies which are intended to selectively kill or reduce the growth, division or viability of tumour or cancer cells compared to non-tumour or non-cancer cells. The invention relates to the fields of molecular biology, cell biology and pharmacology.

In the more affluent countries of the world cancer is the cause of death of roughly one person in five. The American Cancer Society in 1993 reported that the five most common cancers are those of the lung, stomach, breast, colon/rectum and the uterine cervix. Tumour cells have lost the normal control of the cell cycle and so divide out of control compared to normal cells. The sub-cellular machinery which controls cellular processes is made up of a complex biochemical network of interacting proteins that induce and co-ordinate the essential processes of cell growth, duplication and division.

Environmental cues are deciphered by cellular regulatory elements to adjust the metabolic state within the cell to reflect external conditions and maintain cellular homeostasis. The mammalian target of rapamycin (mTOR or TOR) is a member of the phosphatidylinositide kinase related family of protein kinases and resides at the interface between nutrient sensing and the regulation of major metabolic responses (Dennis et al., Curr Opin Genet & Dev 9, 49 (1999); Gingras et al., Genes & Dev. 15, 807 (2001); Schmelzle and Hall, Cell 103, 193 (2000)). More specifically, depending on mitogen and amino acid availability, mTOR positively regulates translation and ribosome biogenesis while negatively controlling autophagy (Dennis et al., 1999), leading to the suggestion that mTOR acts to set protein synthetic rates as a function of the availability of translational precursors (Hara et al., J. Biol. Chem. 273,14484 (1998); liboshi et al., J.Biol.Chem. 274,1092 (1999)). In response to mitogens and amino acids, mTOR phosphorylates and controls the activities of two key translational regulators, S6 Kinase 1 (S6K1) and initiation factor 4E binding protein (4E-BP1) (Gingras et al., 2001). However, the ability to detect changes in mTOR activity in vitro, following either mitogen or amino acid treatment, has been difficult to demonstrate (Gingras, et al., 2001; Schmelzle and Hall, 2000).

The importance of understanding the molecular mechanisms that control mTOR function is underscored by recent Phase 1 clinical trials showing that rapamycin is efficacious in the treatment of solid tumors in patients with metastatic renal cell carcinoma, and non-small cell lung, prostate, and breast cancer (Hidalgo and Rowinsky, 2000, Oncogene 19, 6680). Nevertheless, there remains a need to find alternative ways of selectively killing a wide range of cancer cells whilst leaving normal cells of the body unaffected.

### RELEVANT LITERATURE

P. B. Dennis, S. Fumagalli, G. Thomas, Curr Opin Genet & Dev 9, 49 (1999).
A. C. Gingras, B. Raught, N. Sonenberg, Genes & Dev. 15, 807 (2001).
T. Schmelzle, M. N. Hall, Cell 103, 193 (2000).
K. Hara et al., J. Biol. Chem. 273, 14484 (1998).
M. Hidalgo, E. K. Rowinsky, Oncogene 19, 6680 (2000).
E. V. Schmidt, Oncogene 18, 2988 (1999).
J. Roberts, Science 278, 2073 (1997).
C. J. Lynch, H. L. Fox, T. C. Vary, L. S. Jefferson, S. R. Kimball, J. Cell. Biochem. 77, 234 (2000).
T. Gaal, M. S. Bartlett, W. Ross, C. L. Turnbough, Jr., R. L. Gourse, Science 278, 2092 (1997).

### SUMMARY OF THE INVENTION

The present invention provides a method for screening for a potential modulator of TOR comprising: incubating a test agent with a cell; detecting a decrease in ATP levels in the cell relative to when the test agent is absent; and correlating a decrease in ATP levels in the cell with the presence of a potential modulator of TOR. The decrease in ATP levels is at least 1% preferably, at least 5-10%, but no more than 75%, preferably 50%. ATP levels in a cell can be conventiently detected using luciferase-based assays.

The potential modulator is preferably effective against a disease or condition dependent on mTOR signaling, including without limitation cancer, rheumatoid arthritis, restinosis and transplantation rejection, and therefore can be used for the treatment, including the prophylactic treatment of these diseases and conditions.

The invention therefore also provides compositions for the prevention or prophylactic treatment of tumourigenesis or the treatment or prophylactic treatment of tumours, rheumatoid arthritis (or other inflammatory diseases), restinosis, transplant rejection or the treatment or prophylactic treatment of any disease involving mTOR, comprising a compound that reduces ATP levels in a cell by at least at least 1% preferably, at least 5-10%. The compound preferably does not reduce ATP levels in a cell by more than 75%. The composition may be provided together with a pharmaceutically acceptable excipient, diluent or carrier, for use as a pharmaceutical.

Also encompassed by the invention is the use of such compositions for the manufacture of a pharmaceutical or for the treatment of a disease or condition dependent on mTOR, or for the treatment of cancer, rheumatoid arthritis, restinosis or transplant rejection. The cancer will typically be characterized by having high intracellular ATP concentrations. The cancer may be a solid tumor. The cancer may be epithelial or mesenchymal, such as a glioblastoma or breast carcinoma.

Also provided are methods of treating a disease or condition dependent on mTOR, comprising administering an effective amount of a compound that reduces ATP levels in a cell by at least at least 1% preferably, at least 5-10%, but not more than 75%. The disease or condition can be selected from the group consisting of: cancer, rheumatoid arthritis, restinosis and transplant rejection.

In a further aspect of the invention, a method of diagnosing or prognosing a disease or condition dependent on mTOR is provided. The method comprises obtaining a sample from an individual; analysing the sample for the presence of ATP or an ATP marker (such as a glycolytic enzyme); and correlating the presence of an elevated level of ATP or the ATP marker relative to a sample from an unafflicted individual with an unfavourable prognosis or diagnosis.

### DETAILED DESCRIPTION

The bacterial macrolide, rapamycin is an efficacious anti-cancer agent against solid tumors. In a hypoxic environment the increase in mass of such tumors is dependent on the recruitment of mitogens and nutrients. When nutrient concentrations change, particularly those of essential amino acids, the mammalian Target Of Rapamycin (mTOR), functions in regulatory pathways that control ribosome biogenesis and cell growth. The present invention is based on the observation that the mTOR pathway is influenced by the intracellular concentration of ATP, independent of the abundance of amino acids, and that mTOR itself is an ATP sensor. It is proposed that as ATP is utilized in eukaryotic cells, mTOR functions as a homeostatic sensor, adjusting the rate of ribosome biogenesis to reflect intracellular ATP concentrations.

In tumors metabolic flux is redirected to glycolysis, leading to the more rapid production of ATP. The present inventors believe that such tumours having an increased production of ATP can be more susceptible to the effects of mTOR inhibitors and more sensitive to reduction in intracellular ATP concentrations, compared to normal cells.

Accordingly, the present invention provides a method for screening for a potential modulator of mTOR signalling comprising: incubating a test agent with a cell, detecting a decrease in ATP levels in said cell relative to when said test agent Is absent; and correlating a decrease in ATP levels in said cell with the presence of a potential modulator of mTOR signalling. The cell is preferably a mammalian cell, more preferably a human cell, and typically will be available as a cell line for ease of propagation.

The test agent can be present in a library of compounds, which can be tested in pools to reduce the time needed to identify a potential modulator. Once a potential modulator is identified as being present In a pool of test agents, each individual test agent can be re-tested to identify the potential modulator. Alternatively, known compounds can be tested without pooling and chemically modified to obtain or improve the desired property. For example, known ATP depleting agents include 2-deoxyglucose, cyanine, oligomycin, valinomycin and azide, as well as salts and derivatives thereof. Such ATP depleting agents can be chemically modified or provided in formulations to give the desired effect, i.e., a decrease in intracellular ATP levels sufficient to affect mTOR signaling but without having a detrimental effect on normal cells.

Typically, useful modulators will result in a decrease of at least 1% in intracellular ATP levels, preferably at least 5%, more preferably at least 15% or more. To avoid detrimental effects on normal cells, the decrease is preferably no more than 75%, most preferably no more than 50%.

ATP levels can be determined by any method known in the art or any method yet to be discovered. Examples of methods that can be used to determine ATP levels are described in the Examples below, which use a luciferase assay to detect ATP, as well as in U.S. Pat. No. 5,618,682 and WO 00/18953, which are hereby incorporated by reference in their entirety.

The screening methods of the Invention may further comprise control cells grown in the absence of test agent and ATP levels are measured in both control and test cultures. The test measurements can thereby be normalized with respect to the control. Other internal controls can also be employed to test for reproducibility of the assay or any other desired characteristic, as is well known in the art.

Throughout the assays of the invention, incubation and/or washing steps may be required after each application of reagent or incubation of combinations of reagents. Incubation steps may vary from about 5 minutes to several hours, perhaps from about 30 minutes to about 6 hours. However, the incubation time usually depends upon the assay format, analyte, volume of solution, concentrations, and so forth. The assays can be carried out at ambient temperature, although they may also be conducted at temperatures in the range of 4° C. to 40° C., for example. Assays with cell extracts are typically carried out at 4° C.

mTOR is known to play a pivotal role in a number of diseases and conditions. Thus, the potential modulator can be further tested for its effectiveness against any disease or condition dependent on mTOR signaling, such as by determining the effect of the potential modulator on mTOR kinase activity. The disease or condition includes without limitation cancer, rheumatoid arthritis (or other inflammatory diseases), restinosis (endothelial cell inhibition) and transplantation rejection. Thus, also included within the scope of the present invention are potential modulators of mTOR signaling for the treatment or prophylactic treatment of cancer, rheumatoid arthritis, restinosis and prevention of transplant rejection or for the treatment or prophylactic treatment of any disease or condition involving mTOR signalling, identified by any of the screening methods of the invention. These substances may be proteins, polypeptides, natural compounds (e.g., polyketides) or small organic molecules (drugs). The invention therefore includes pharmaceutical compositions for preventing or treating any disease or condition involving mTOR signalling comprising one or more of the substances identified by a screening method of the invention.

Thus, in a further aspect of the invention, compositions are provided, in particular pharmaceutical compositions for humans or veterinary compositions for animals, for the prevention or prophylactic treatment of tumourigenesis or the treatment or prophylactic treatment of tumours (including mesenchymal or epithelial tumours, such as, without limitation, glioblastoma, or leukemia (in particular abnormal T cell proliferation), lung, stomach, breast, colon/rectum, uterine or cervical cancer), rheumatoid arthritis, restinosis, prevention of transplant rejection or the treatment or prophylactic treatment of any disease involving mTOR signalling, comprising a compound that reduces ATP levels in a cell by at least at least 1% preferably, at least 5-10%, or at least 25% but not more than 75%, preferably not more than 50%. The compositions preferably comprise a compound that affects mTOR activity. Thus, the compositions will typically be effective against cancers (e.g., solid tumours or cancers with high intracellular ATP concentrations) that are rapamycin sensitive. The compositions may also include other active or non-active agents. Non-active agents may include a pharmaceutically acceptable excipient; diluent or carrier, such as saline, buffered saline, dextrose or water.

The present invention further provides the use of a potential modulator of mTOR signalling as hereinbefore, for the manufacture of a medicament for the prevention or prophylactic treatment of a disease or condition dependent on mTOR signaling, such as cancer, rheumatoid arthritis, restinosis or prevention of transplantation rejection (immunosuppression).

The compositions and medicaments of the invention may therefore be used prophylactically in order to prevent tumours or other diseases from developing, or they may be used in a curative or partly curative way to treat or contain a pre-existing condition. The tumours or tumour cells are preferably those which are sensitive to rapamycin and/or those which exhibit a high intracellular ATP concentration. In particularly preferred embodiments the tumours are solid tumours, e.g. mesenchymal tumours such as glioblastoma or epithelial cancers such as breast carcinoma.

The invention also provides a method of treating a disease or condition dependent on mTOR, comprising administering an effective amount of a compound that reduces ATP levels in a cell by at least at least 1% preferably, at least 5-10%, at least 25%, but not more than 75%, preferably not more than 50%. The term “treating” is meant to encompass prophylactic treatment as well as the treatment of an existing disease or condition. The disease or condition includes without limitation, cancer, rheumatoid arthritis, restinosis and prevention of transplant rejection.

The determination of an effective dose Is well within the capability of those skilled in the art. For any compound, the therapeutically effective dose can be estimated initially either in cell culture assays or in an appropriate animal model. The animal model is also used to achieve a desirable concentration range and route of administration. Such information can then be used to determine useful doses and routes for administration in humans.

A therapeutically effective dose refers to that amount of active agent which ameliorates the symptoms or condition. Therapeutic efficacy and toxicity of such compounds can be determined by standard pharmaceutical procedures in cell cultures or experimental animals (e.g., ED50, the dose therapeutically effective in 50% of the population; and LD50, the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage may be chosen by the individual physician in view of the patient to be treated. Dosage and administration can be adjusted to provide sufficient levels of the active moiety or to maintain the desired effect. Additional factors which may be taken into account include the severity of the disease state (e.g. tumour size and location); age, weight and gender of the patient; diet; time and frequency of administration; drug combination(s); reaction sensitivities; and tolerance/response to therapy. Long acting pharmaceutical compositions can be administered on a daily basis, every 3 to 4 days, every week, or once every two weeks, depending on half-life and clearance rate of the particular formulation.

Administration of pharmaceutical compositions of the invention may be accomplished orally or parenterally. Methods of parenteral delivery include topical, intra-arterial (e.g. directly to the tumour), intramuscular, subcutaneous, intramedullary, intrathecal, intraventricular, Intravenous, intraperitoneal, or intranasal administration. In addition to the active ingredients, these pharmaceutical compositions can contain suitable pharmaceutically acceptable carriers comprising excipients and other compounds that facilitate processing of the active compounds into preparations which can be used pharmaceutically. Further details on techniques for formulation and administration can be found in the latest edition of Remington's Pharmaceutical Sciences (Maack Publishing Co, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, etc, suitable for ingestion by the patient.

Pharmaceutical preparations for oral use can be obtained through combination of active compounds with solid excipient, optionally grinding a resulting mixture, and processing the mixture of granules, after adding suitable additional compounds, if desired, to obtain tablets or dragee cores. Suitable excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose., or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores can be provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterise the quantity of active compound (i.e. dosage).

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds can be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention can be manufactured in substantial accordance with standard manufacturing procedures known in the art (e.g. by means of conventional mixing, dissolving, granulating, dragee-making, levigating, emulsifying, encapsulating, entrapping or lyophilising processes).

The present invention also provides a method of diagnosing or prognosing a disease or condition dependent on mTOR, comprising: obtaining a sample from an individual; analysing the sample for the presence of a marker indicative of ATP levels in the sample; and correlating the presence (or an increase in the level of the marker relative to a value obtained from an unafflicted individual) with an unfavourable prognosis or diagnosis. Such a marker is easily selected by one of ordinary skill in the art and includes glycolytic enzymes or their activity, for example. A statistically significant increase in the presence of such a marker, or its activity, relative to normal tissue would therefore indicate an unfavourable prognosis or diagnosis. The intracellular ATP concentration could itself act as such a marker and as an indicator of an individual having a higher risk of a disease or condition dependent on mTOR signaling.

In a preferred embodiment, the invention provides kits suitable for use in the diagnostic or prognostic methods of the invention. Such kits comprise reagents useful for carrying out these methods, for example, an antibody specific for the marker (e.g., a glycolytic enzyme), reagents useful in detecting glycolytic enzymatic activity, or detecting ATP, such as luciferase.

Preferred embodiments of the invention will now be described by way of example, which should not be considered limiting in any way.

### EXAMPLES

### Example 1

### mTOR is Sensitive to Metabolic Inhibitors

Because mTOR is sensitive to amino acids and regulates ribosome biogenesis (Dennis et al., 1999; Gingras et al., 2001; Schmelzle and Hall, 2000), we tested whether its activity may be sensitive to metabolic inhibitors. We used, insulin-induced S6K1 activation and 4E-BP1 phosphorylation as reporters for mTOR function in the presence of glycolytic or mitochohdrial inhibitors to reduce ATP production.

Briefly, human embryonic kidney cells (HEK293) were seeded and maintained as previously described (Pullen et al., Science 279, 707 (1998)). Confluent cells were serum starved for 20 h and then extracted (controls) or treated with 200 nM insulin in the precence or absence of 100 mM 2-deoxyglucose or 20 mM rotenone for 30 min. Cell extraction, kinase assays and Western blot analysis (used to measure S6K1 levels and T389 phosphorylation) were performed as described (Pullen et al.,1998). Phosphospecific antibodies are commercially available (e.g., New England Biolabs). Although an inhibitory effect was seen with rotenone, the glycolytic inhibitor 2-deoxyglucose (2-DG) was shown to be more effective in inhibiting S6K1 T389 phosphorylation and S6K1 activation (determined by detecting S6 phosphorylation) than the mitochondrial inhibitor, rotenone, reducing phosphorylation/activation to background levels.

The results were confirmed using a different target of mTOR, namely 4EBP-1. Transfections were carried out essentially as described by Manteuffel et al., 1997, Mol Cell. Biol. 17, 5426 with the 4EBP-1 construct described therein. The expression and phosphorylation of transiently-transfected HA-4E-BP1 was measured using a polyclonal anti-HA antibody and phosphospecific antibody against S65, respectively, essentially as described above (Pullen et al., 1998). The anti-HA antibody was obtained from a commercial source (Santa Cruz Biotechnology, Inc). The mTOR-mediated phosphorylation site in 4E-BP1 is S65. Western blot analysis showed an inhibitory effect of rotenone on S65 phosphorylation in insulin stimulated cells, whereas the glycolytic inhibitor 2-deoxyglucose (2-DG) was again more effective, as demonstrated by its ability to inhibit 4EBP-1 S65 phosphorylation to background levels. Similar results were obtained when phosphorylation of the S65 site was measured after treatment with iodoacetic acid or dinitrophenol, known glycolytic or mitochondrial inhibitors, respectively (i.e., iodoacetic acid was found to be more efficient than dinitrophenol).

In summary, the metabolic inhibitors iodoacetic acid, dinitrophenol, 2-deoxyglucose and rotenone are all able to inhibit phosphorylation of mTOR targets. However, only the glycolytic inhibitors (2-deoxyglucose and lodoacetic acid) are able to reduce phosphorylation of mTOR targets to background levels.

### Example 2

### mTOR is Sensitive to Alterations in Intracellular ATP

Because mTOR is sensitive to glycolitic inhibitors, we wanted to determine whether mTOR is sensitive to alterations in intracellular ATP concentrations. ATP levels were measured from mock-transfected HEK293 cells (using empty vector) treated as in Example 1, using a luciferase-based assay. Retonone and 2-deoxyglucose treatments were carried out on insulin-stimulated cells.

To generate extracts for ATP assays, cells were washed twice with 10 ml ice cold PBS, drained thoroughly, scraped into 1 ml of buffer (100 mM Tris-HCl and 4 mM EDTA pH 7.75) and transferred into an Eppendorf tube before flash freezing in liquid nitrogen. The frozen cells were boiled for 3 min and then placed on ice for 5 min followed by centrifugation at 13,000 rpm for 5 min at 4° C. ATP levels in the extract were measured in a microtiter plate by a Luciferase-based assay (Roche, ATP Bioluminescence Assay Kit CLS II) using a Microlumat LB96P microtiter plate reader (EG&G Berthold). In an illustrative set of experiments and expressing results as a percentage of the insulin-stimulated control (100%), ATP levels increased slightly in insulin treated cells (94% in unstimulated cells), decreased in 2-deoxyglucose treated cells (40-49%) and rotenone-treated cells (75-83%). The ability of each agent to lower ATP concentrations therefore paralleled its effectiveness in inhibiting S6K1 activation suggesting that mTOR is sensitive to alterations in intracellular ATP.

### Example 3

### Selectivity of Metabolic Inhibitors

The modest effect of rotenone in reducing ATP concentrations, as compared to its effects on S6K1 and 4E-BP1 phosphorylation, suggested the metabolic inhibitors were not generally toxic. To verify this, the effect of 2-deoxyglucose was tested on Protein Kinase B (PKB) and Mitogen-Activated Protein Kinase (MAPK).

Transiently-transfected, HA-tagged PKB activation was measured in vitro using histone 2B (H2B) as substrate (Franke et al., 1995, Cell 81, 727) after immunoprecipitation from serum-starved cells extracted directly or after insulin stimulation with or without the addition of 100 mM 2-deoxyglucose, 20 nM rapamycin or 100 nM wortmannin. Rapamycin and wortmannin were added to serum-starved cells 30 min prior to insulin-stimulation. HA-PKB expression and S473 phosphorylation were measured essentially as described above. HA-MAPK kinase activity toward using myelin basic protein (MBP) was measured from serum-starved cells extracted directly or after 100 nM TPA stimulation in the presence or absence of 20 nM rapamycin or 100 mM 2-deoxyglucose for 30 min (N. Pullen et al., 1998).

Like rapamycin, 2-deoxyglucose did not inhibit insulin-induced activation of PKB, as judged by S473 phosphorylation and in vitro phosphorylation of H2B. However, both S473 and H2B phosphorylation were shown to be sensitive to wortmannin, a phosphatidylinositide-3OH kinase (PI3K) inhibitor. In addition, 2-deoxyglucose had no discernable effect on TPA-induced MAPK activation, as judged by MBP phosphorylation. Hence, the effect of 2-deoxyglucose in reducing ATP concentrations is selective for signaling to S6K1 and 4E-BP1.

### Example 4

### Specific Inhibition of mTOR-Signalling by 2-deoxyglucose

To assess whether the inhibitory effects of 2-deoxyglucose on S6K1 activation were mediated by mTOR, we used a rapamycin-resistant allele of S6K1. Rapamycin resistance was conferred by fusing glutathione-S-transferase to the NH₂-terminus of S6K1 and truncating the COOH-terminus, creating a construct termed GST-ΔC-S6K1 (Pullen et al.,1 998). For the construction of the GST-ΔC-S6K1 (also known as GST-ΔC₁₀₄-S6K1) and D3E, K100Q-S6K1-GST plasmids, similar cloning strategies were used. S6K1-GST or GST-ΔC-S6K1 from transiently-transfected, serum-starved HEK293 cells were extracted directly or after insulin stimulation with or without the addition of 20 nM rapamycin or 100 nM wortmannin. Kinase activities, expression levels and T389 phosphorylation were carried out essentially as described above. Expression and activity of S6K1-GST or GST-ΔC-S6K1 were measured after insulin stimulation alone or with increasing amounts of 2-deoxyglucose (20, 40 and 100 mM) essentially as described above. Mock-transfected HEK293 cells were treated using the same conditions and extracted for ATP analysis

Both S6K1, having a COOH-terminal GST tag (S6K1-GST), and GST-ΔC-S6K1 were phosphorylated and activated by insulin in a wortmannin-sensitive manner, however only GST-ΔC-S6K1 was resistant to inhibition by rapamycin. 2-deoxyglucose reduced insulin-induced activation of S6K1-GST (rapamycin sensitive) in a dose-dependent manner, paralleling its effect on intracellular ATP concentrations. Compared to ATP levels in an Insulin stimulated control (100%), ATP levels decreased to 57-59%, 43-48% and 32-36% after treatment of S6K1-GST transfected cells with 20, 40 and 100 nM 2-deoxyglucose, respectively. In contrast, insulin-induced activation of GST-ΔC-S6K1 (rapamycin-resistant) was unaffected by 2-deoxyglucose treatment. Thus, 2-DG appears to selectively inhibit signaling to mTOR effectors, supporting a model whereby mTOR is controlled by intracellular ATP concentrations.

### Example 5

### lntracellular ATP Levels have a Direct Effect on mTOR Activity

Because mTOR signaling Is dependent on concentrations of aminoacylated tRNAs (liboshi et al., 1999, J.Biol.Chem. 274,1092), the effects of ATP on S6K1 and 4E-BP1 may be indirect, occurring through inhibition of tRNA aminoacylation. To examine this possibility, we analyzed total cellular tRNA on acid-urea polyacrylamide gels, which resolve aminoacylated from non-acylated tRNA.

Briefly, total RNA was isolated from serum-starved HEK293 cells directly or after insulin stimulation with or without 2-deoxyglucose or amino acid starvation. As a control marker, tRNA was deacetylated by mild alkaline hydrolysis in 0.1 M Tris-HCl (pH 8.0) at 75° C. for 5 min. After resolution on an acid/urea gel (Enriquez et al., in Methods Enzymol., Acad. Press, San Diego, vol. 264, pp. 183.), tRNA was visualized with ethidium bromide staining. Expression levels and phosphorylation state of endogenous S6K1 and 4E-BP1 were also measured from serum-starved cells, stimulated with insulin in the presence or absence of amino acids in the media essentially as described in Example 1.

Neither insulin stimulation nor 2-deoxyglucose treatment had an effect on total amounts of aminoacylated tRNA. Unexpectedly, amino acid deprivation also had no effect, even though such treatment was sufficient to completely block phosphorylation of S6K1 and 4E-BP1.

To further test this finding, selected tRNAs were examined by Northern blot analysis of the tRNA resolved by acid/urea gel electrophoresis as described above. The individual tRNA species were probed after electrotransfer to nylon membrane, with radiolabelled oligonucleotides specific for tRNALeu and tRNAHis, namely: tRNALeu 5′-GCG CCT TAG ACC GCT CGG CCA CG-3′ (SEQ ID NO:1); tRNAHis 5′-GGT GCC GTG ACT CGG ATT CGA ACC G-3′ (SEQ ID NO:2); and tRNAThr 5′-GCG AGA AAT GM CTC GCG-3′ (SEQ ID NO:3). As with total tRNA, none of the treatments had an effect on the aminoacylation status of leucyl, histidyl or threonyl tRNA, indicating that amino acid pools, rather than amounts of aminoacylated tRNA, are important for mTOR signaling.

Levels of individual amino acids were measured in extracts prepared from insulin-stimulated HEK293 cells in the presence or absence of amino acids or with 100 mM 2-deoxyglucose treatment and expressed as a percentage of the insulin-stimulated control in the presence of amino acids. Briefly, confluent cultures of HEK293 cells were treated as described above, washed with PBS and drained thoroughly. The cells were then scraped into 500 ml water and sonicated with four 10 sec pulses. Cell debris was removed by centrifugation and sulfosalicylic acid was added to the supernatant to a final concentration of 2%. The samples were then placed on ice for 30 min followed by centrifugation to remove precipitated proteins. The extracts were then analyzed for amino acid content with a Biochrom 20 plus amino acid analyzer (Amersham-Pharmacia Biotech).

Amino acid deprivation was demonstrated to result in a decrease in the amounts of essential amino acids, particularly the branched-chain amino acids. For example, Leu, Ile and Val levels decreased to 10-20% of the control. In contrast, 2-deoxyglucose had little effect on amino acid levels (Ala, Glu, Leu, Ile and Val, all 100-120%). Furthermore, amino acid deprivation had no effect on concentrations of ATP. Thus, regulation of mTOR by ATP is independent of amino acids pools.

### Example 6

### mTOR Activity Requires High ATP Concentrations.

Reducing ATP concentrations could lead to a stable change in mTOR activity, through a post-translational modification, or ATP could directly affect mTOR activity. To test the first possibility, we transiently expressed HA epitope-tagged mTOR in HEK293 and measured its activity after treatment of cells with 2-deoxyglucose.

Briefly, transiently transfected, hemaglutinin-tagged (HA) wild-type mTOR (HA-mTORwt) from HEK293 cells were serum starved for 16 hours before insulin stimulation. The cells were then treated with (or controls without) 100 mM 2-deoxyglucose or subjected to amino acid starvation as described above. Cell extracts were prepared for immunoprecipitations with a monoclonal anti-HA antibody. The immunocomplex was washed once with 1 M NaCl in assay buffer (30 mM MOPS (pH7.5), 5 mM NaF, 20 mM β-glycerol phosphate, 1 mM dithioerythritol, 0.1% Triton X-100 and 10% glycerol) and twice with assay buffer alone. HA-mTORwt was assayed for T389 kinase activity, using a kinase inactive mutant of S6K1 (D3E, Pearson et al., 1995, EMBO J. 14, 5279; K100Q-GST, von Manteuffel et al., 1997, Mol. Cell. Biol. 17, 5426) as substrate. One microgram of a kinase inactive, purified, soluble S6K1 substrate (S6K1-D3E,K100 Q-GST) was added along with assay buffer containing 10 mM MgCl₂ and 1 mM ATP and incubated for 30 min at 30° C. Quantitation for Km measurements was carried out using scanning densitometry and ImageQuant software (Molecular Dynamics).

To test whether an activated allele of P13K could affect mTOR activity in vitro, HA-mTORwt was transiently expressed alone or with a constitutively membrane-targeted phosphatidylinositide-3-OH kinase (CD2-P13K, Reif et al., 1997, J. Biol. Chem. 272, 14426) using standard techniques and then extracted directly or stimulated with insulin before extraction essentially as described above.

Although 2-deoxyglucose treatment blocked insulin-induced activation of S6K1 (see Example 1), it had no effect on the kinase activity of mTOR in vitro towards T389 of S6K1. Indeed, amino acid deprivation, insulin stimulation, or transient expression of an activated-allele of P13K had no effect on mTOR kinase activity in vitro. Thus, it seems unlikely that a post-translational modification directly affects mTOR activity.

As a control, Myc-tagged, wild-type S6K1 was expressed alone or with CD2-P13K in starved or insulin-stimulated HEK293 cells. S6K1 kinase activity was assayed as in Example 1 after immunoprecipitation with an anti-myc antibody and Cd2-P13K, as well as insulin, CD″-P13K was shown to be able to induce T389 phosphorylation and S6K1 activation in intact cells.

Next, we measured mTOR activity in vitro at ATP concentrations that approached physiological levels of 1-5 mM In mammalian cells (Gribble et al., 2000, J.Biol.Chem. 275, 30046). The expression and activities of HA-mTORwt and inactive mTOR kinase were assayed using S6K1 (T389 phosphorylation) or 4E-BP1 (S65 phosphorylation) as substrates essentially as described above. ATP concentrations used in the assay were 3.0 mM for inactive kinase and 0.2, 0.4, 0.8, 1.6, 2.3 and 3.0 mM for wild-type mTOR.

Specific activity of mTOR for S6K1 T389 phosphorylation increased up to ˜1 mM ATP, saturating at around 2-3 mM ATP, whereas the catalytically inactive mTOR mutant did not phosphorylate T389. On the basis of these values we calculated an apparent Km for ATP of at least 1 mM. Most protein kinases analyzed to date show an apparent Km for ATP of 10-20 mM (Edelman et al., in Annu.Rev.Biochem., Annual Reviews Inc., Palo Alto, 1987, vol. 56, pp. 567), one fiftieth to one hundredth of that observed for mTOR. Because mTOR also phosphorylates several sites in 4E-BP1, including S65, we also assayed the ATP requirement of mTOR for S65 of 4E-BP1. Using the same assay conditions described for T389 phosphorylation in S6K1, we obtained almost identical results for S65 phosphorylation. These findings sustain the role of mTOR as an ATP effector, and suggest it is a direct sensor of ATP in the cell. Therefore, intracellular concentrations of ATP directly regulates mTOR, whereas amino acids employ a separate mechanism

Without wishing to be bound by theory, the inventors propose that as ATP is utilized in eukaryotic cells, mTOR functions as a homeostatic sensor, adjusting the rate of ribosome biogenesis to reflect intracellular ATP concentrations. Interestingly, increased ribosome biogenesis is a predictive indicator of tumor progression (Derenzini et al., 2000, J. Pathol. 191, 181) and in tumors metabolic flux is redirected to glycolysis, leading to the more rapid production of ATP (Pfeiffer et al., 2001, Science 292, 504 (2001); Dang et al., 1999, Trends Biochem. Sci. 24, 68). If such tumors gain an mTOR-specific growth advantage, due to increased production of ATP, they may be more susceptible to the effects of mTOR inhibitors. Because of the low Km of mTOR for ATP (1 mM), tumours may also be more sensitive to reduction in cellular ATP levels. In contrast, most cellular kinases have a Km for ATP in the range of 10-20 μM and would remain unaffected by marginally reduced intracellular ATP concentrations.

The disclosures of each publication referred to herein are hereby incorporated by reference in their entireties, as if each reference were referred to individually.

1. A method for screening for a potential modulator of TOR comprising:
(a) incubating a test agent with a cell;
(b) detecting a decrease in ATP levels in said cell relative to when said test agent is absent; and
(c) correlating a decrease in ATP levels in said cell with the presence of a potential modulator of TOR.

2. The method of claim 1, wherein said decrease is at least 1% preferably, at least 105-10%.

3. The method of claim 1, wherein said decrease is no more than 50%.

4. The method of claim 1, wherein said ATP levels in said cell are detected using a luciferase assay.

5. The method of claim 1, wherein said potential modulator is effective against a disease or condition dependent on mTOR signaling.

6. The method of claim 1, wherein said potential modulator is effective against any one of the diseases or conditions selected from the group consisting of: cancer, rheumatoid arthritis, restinosis and transplantation rejection.

7. A composition for the prevention or prophylactic treatment of tumourigenesis or the treatment or prophylactic treatment of tumours, rheumatoid arthritis, restinosis, transplant rejection or the treatment or prophylactic treatment of any disease involving mTOR, comprising a compound that reduces ATP levels in a cell by at least at least 1% preferably, at least 5-10%, but not more than 75%.

8. The composition of claim 7, further comprising a pharmaceutically acceptable excipient, diluent or carrier.

9. The composition of claim 7, wherein said compound affects mTOR activity.

10. The composition of claim 7, for use as a pharmaceutical.

11. The use of a composition of claim 7 for the treatment of cancer, rheumatoid arthritis, restinosis or transplant rejection.

12. The use as claimed in claim 11, wherein the cancer is rapamycin sensitive.

13. The use as claimed in claim 12, wherein the cancer is characterized by a high intracellular ATP concentration.

14. The use as claimed in claim 12, wherein the cancer is mesenchymal or epithelial.

15. The use as claimed in claim 12 wherein the cancer is a solid tumour.

16. The use as claimed in claim 12, wherein the cancer is a glioblastoma or breast carcinoma.

17. A method of treating a disease or condition dependent on mTOR, comprising administering an effective amount of a compound that reduces ATP levels in a cell by at least at least 1% preferably, at least 5-10%, but not more than 75%.

18. The method of claim 17, wherein said disease or condition is selected from the group consisting of: cancer, rheumatoid arthritis, restinosis and transplant rejection.

19. A method of diagnosing or prognosing a disease or condition dependent on mTC3R, said method comprising: a) obtaining a sample from an individual; b) analysing said sample for the presence ATP or an ATP marker; and c) correlating the presence of an elevated level of said ATP or ATP marker relative to a sample from an unafflicted individual with an unfavourable prognosis or diagnosis.

20. The method of claim 19, wherein said marker is a glycolytic enzyme.


SEQUENCE LISTING (3 sequences)

SEQ ID NO: 1 - DNA, 23 bp, Homo sapiens
  tRNA (1)...(23): Oligonucleotide specific for tRNALeu
gcgccttagaccgctcggccacg

SEQ ID NO: 2 - DNA, 25 bp, Homo sapiens
  tRNA (1)...(25): Oligonucleotide specific for tRNAHis
ggtgccgtgactcggattcgaaccg

SEQ ID NO: 3 - DNA, 18 bp, Homo sapiens
  tRNA (1)...(18): Oligonucleotide specific for tRNAThr
gcgagaattgaactcgcg